(19) United States
(12) Patent Application Publication
(10) Pub. No.: US 2025/0161186 A1
(43) Pub. Date: May 22, 2025

(54) LIQUID BOTULINUM TOXIN FORMULATION AND USE THEREOF

(71) Applicant: MERZ PHARMA GMBH & CO. KGAA, Frankfurt am Main (DE)

(72) Inventors: Karl-Heinz EISELE, Frankfurt am Main (DE); Klaus FINK, Frankfurt am Main (DE); Harold V. TAYLOR, Frankfurt am Main (DE); Claudia JATZKE, Frankfurt am Main (DE); Katja KÜHBACH, Frankfurt am Main (DE)

(73) Assignee: MERZ PHARMA GMBH & CO. KGAA, Frankfurt am Main (DE)

(21) Appl. No.: 18/838,546

(22) PCT Filed: Feb. 14, 2023
(86) PCT No.: PCT/EP2023/053634

§ 371 Date: Aug. 14, 2024

(30) Foreign Application Priority Data
Feb. 15, 2022 (EP) 22156850.4

(51) Int. Cl. A61K 8/66; A61K 8/27; A61K 8/44

(52) U.S. Cl. CPC A61K 8/66; A61K 8/27; A61K 8/44; A61K 2800/51; C12Y 304/24069

(57) ABSTRACT

The present invention relates to a liquid botulinum toxin formulation comprising: (i) botulinum toxin, (ii) a stabilizing protein such as human serum albumin, (iii) a chelating agent (e.g., EDTA) or phosphate, and optionally (iv) a salt of calcium, magnesium or zinc, (v) a tonicity agent and/or (vi) a buffering agent. In addition, the present invention relates to the use of the liquid botulinum toxin formulation for therapeutic and cosmetic treatments.

### FIELD OF THE INVENTION

The present invention relates to a liquid botulinum toxin formulation comprising: (i) botulinum toxin, (ii) a stabilizing protein such as human serum albumin, (iii) a chelating agent (e.g., EDTA) or phosphate, and optionally (iv) a salt of calcium, magnesium or zinc, (v) a tonicity agent and/or (vi) a buffering agent. In addition, the present invention relates to the use of the liquid botulinum toxin formulation for therapeutic and cosmetic treatments.

### BACKGROUND OF THE INVENTION

Botulinum neurotoxins (BoNTs; or botulinum toxin (BT)) are a family of bacterial neurotoxins that are widely used to treat a growing variety of neurologic, medical and cosmetic conditions. There are eight serotypes of BoNTs (BoNT/A-H). Two serotypes, type A (BoNT/A) and type B (BoNT/B), are currently in clinical use. BoNTs are produced by Clostridium spp., in particular Clostridium botulinum, in the form of high molecular weight (up to ˜900 kDa) complexes. These toxin complexes are composed of the active 150 kDa neurotoxin and several complexing proteins (nontoxic neurotoxin-associated proteins, NAPs).

The 150 kDa neurotoxin is synthesized as an inactive single chain polypeptide (˜150 kDa), which is proteolytically cleaved to give a light chain (LC, ˜50 kDa) and a heavy chain (HC, ˜100 kDa) connected by an inter-chain disulfide bond. The HC contains a C-terminal domain that mediates binding to receptors and an N-terminal domain that mediates translocation of the LC across endosomal membranes. The LC acts as a protease in neurons and cleaves neuronal SNARE proteins. This blocks the fusion of synaptic vesicles with the plasma membrane, thus inhibiting neurotransmitter release from selected neurons.

The formulation of BoNTs is very challenging, owing to their structural complexity and low product concentrations used. BoNT is highly susceptible to various conditions such as heat and alkaline pH. Thus, since BONT is only functional if its structure is intact, the challenge regarding the preparation of a medical dosage form of BoNT is to formulate a composition that protects the BoNT from inactivation or partial loss of biological activity during production, storage or use of the product. Furthermore, due to the extremely high potency of BoNT, with the human lethal dose being in the range of only about 0.1-1 ng/kg, pharmaceutical formulations of BONT comprise extremely small quantities of the toxin in the range of only about 1 ng per vial. This aggravates the known problem of loss of toxin activity due to surface denaturation. Therefore, one of the main challenges in the formulation of BoNTs is minimizing activity loss during manufacture and storage.

Due to these contributing factors, the main BONT products currently available are provided as lyophilized powders, i.e., in a form that is stable over a long period of time when stored at 2-8° C. or even at room temperature (Xeomin®). Lyophilized forms of the BoNT/A complex were first introduced on the market in 1989 (Botox®, Allergan) and 1991 (Dysport®, Ipsen). In 2005, the first stable dosage form of the pure 150 kDa BoNT/A neurotoxin without complexing proteins was approved (Xeomin®; Merz Pharmaceuticals). However, these lyophilized products need to be reconstituted prior to use, a process potentially leading to dosing errors and sterility issues. Therefore, major efforts have been made to develop liquid formulations of BoNTs, which are more convenient to use and can be easily administered.

The first liquid formulation of BoNT was approved in 2000 and launched in Europe in 2001 as Neurobloc®. Neurobloc® (Eisai) is a sterile solution of the BoNT/B complex formulated in a buffer containing disodium succinate, sodium chloride, human serum albumin (HSA), sodium caprylate, and sodium N-acetyltryptophanate. However, this product may be painful when injected due to its acidic pH. For BoNT/A, only two liquid formulations have obtained market approvals so far: Innotox® (Medytox), but limited to Asian domestic markets (e.g., Korea, approval in 2013), and Alluzience® (Ipsen/Galderma) which was approved for use in Europe in 2021. Both these liquid formulations contain the BoNT/A toxin complex and, in addition to water, sodium chloride, a detergent (Innotox®: polysorbate 20; Alluzience®: polysorbate 80), an amino acid (Innotox®: methionine; Alluzience®: histidine) and an additional excipient (Innotox®: sodium phosphate as a buffer; Alluzience®: sucrose).

Despite these advances in the preparation of liquid botulinum toxin formulations, there is still a need for new options to develop ready-to-use liquid formulations of botulinum toxin, which are stable during transport and storage.

### OBJECT OF THE INVENTION

It is an object of the present invention to provide a stable liquid formulation of botulinum toxin that is ready-to-use for therapeutic and cosmetic treatments.

### SUMMARY OF THE INVENTION

The present invention is based on the surprising finding that the addition of a chelating agent (e.g., EDTA) or phosphate to a human serum albumin (HSA)-containing liquid formulation of botulinum toxin results in an improved formulation that is both heat and light stable.

Thus, in one aspect, the present invention provides a liquid formulation comprising:
(i) botulinum toxin,
(ii) a stabilizing protein, preferably human serum albumin (HSA), and
(iii) a chelating agent or phosphate.

Optionally, the liquid formulation of the present invention further comprises (iv) a salt of calcium, magnesium or zinc, or a mixture thereof, or (v) a tonicity agent (e.g., sodium chloride), or (vi) a buffering agent (e.g., a phosphate buffer or a histidine buffer or both), or two of components (iv)-(vi) (e.g., (iv) and (v), (iv) and (vi), or (v) and (vi)), or all three of components (iv)-(vi).

In another aspect, the present invention relates to a liquid formulation according to the present invention for use in therapy, in particular for use in the treatment of neuromuscular diseases, pain, sialorrhea, hyperhidrosis, urological disorders, and neurological disorders.

In a further aspect, the present invention relates to the cosmetic (aesthetic) use of the liquid formulation of the present invention for the treatment of a cosmetic condition, preferably a skin condition, in particular for the treatment of skin lines or folds or wrinkles.

In yet another aspect, the present invention relates to a method of treating a disease or condition, comprising administering an effective amount of the liquid formulation of the present invention to a person in need thereof.

Preferred embodiments of the liquid botulinum toxin formulation, its use and methods of use according to the present invention are set forth in the appended claims.

The present invention may be understood more readily by reference to the following detailed description of the invention and the example included therein.

### DETAILED DESCRIPTION OF THE INVENTION

The present invention is based on the unexpected finding that the presence of a chelating agent (e.g., EDTA) or phosphate in a human serum albumin (HSA)-containing liquid formulation of botulinum toxin results in an improved formulation that is not only heat but also light stable. In addition, it was unexpectedly found that the addition of a salt of calcium, magnesium, or zinc results in reduced injection pain.

The liquid formulation of the present invention advantageously provides an improved safety and dosing accuracy compared to lyophilized botulinum toxin preparations in powder form since the formulation does not need to be reconstituted prior to injection but is ready-to-use. Furthermore, the liquid formulation exhibits an excellent stability, thereby facilitating transport and storage as well as the handling by the physician.

Without being bound by theory, it is believed that the stabilizing protein, e.g., HSA, is involved in the observed light sensitivity. It is further believed that the surprising finding that the liquid formulation of the present invention has an improved light stability, or in other words exhibits a reduced light-induced loss of toxin activity (reduced photosensitivity), is due to the chelating agent(s) or phosphate which interact with, or exert an effect on, HSA or components that are contained in the employed HSA material, such that the adverse effect of light on the HSA's ability to stabilize botulinum toxin is avoided or minimized.

The improved light stability of the liquid botulinum toxin formulation of the present invention simplifies the manufacturing process, allows the product to be filled and packaged without extensive light protection, and reduces potential loss of activity due to light-exposed storage at the physician's place prior to use. Overall, the reduced sensitivity of the liquid formulation of the present invention generally improves handleability and ease of use of botulinum toxin during injection treatments.

In one aspect, the present invention relates to a liquid formulation comprising
(i) botulinum toxin,
(ii) a stabilizing protein, and
(iii) a chelating agent or phosphate.

As used herein, the term “comprising”, like the terms “including” and containing”, and any variations thereof such as “comprises”, “includes” and “contains”, are intended to refer to a non-exclusive inclusion, such that a process, method, product-by-process, composition or formulation that comprises, includes, or contains an element or list of elements does not include only those elements but can include other elements not expressly listed for such process, method, product-by-process, composition or formulation. In addition, within the framework of the present invention, it is intended that each of the terms “comprise,” “comprising”, “includes”, “including”, “contains”, “containing”, and any variations thereof, can be replaced by the term “consists” or “consisting”, or any variation thereof (e.g., “consists essentially of”), which will be understood to refer to an exclusive inclusion of the elements indicated.

The terms “a”, “an”, “the” and similar reference used in the context of the present invention are to be construed to cover both the singular and the plural and, thus, may also relate to “at least one” or “more than one”, unless otherwise indicated herein or clearly contradicted by the context.

The term “liquid formulation”, as used herein, generally refers to an aqueous formulation and is typically an aqueous solution. Herein, the term “liquid formulation” may be interchangeably used with “liquid composition”. Preferably the liquid formulation is a pharmaceutically acceptable liquid formulation. The term “pharmaceutically acceptable” as used herein, means that the liquid formulation does not cause unacceptable adverse side effects when administered to a human patient or subject. i.e., it means that the liquid formulation is suitable for human use. The aqueous solution may be a buffered solution, with or without a saline solution, and may be a physiological saline solution such as a buffered (e.g., phosphate and/or histidine buffered) physiological saline solution.

The liquid formulation of the present invention can be stored in any suitable container system. A suitable container system for storing the liquid formulation of the present invention is any device having a partially or fully enclosed space that can be sealed or is sealed and can be used to contain, store, and/or transport liquid formulations. A container system is preferably a closed (or sealed) container made of, or partially or predominantly made of, glass or plastic (e.g., organic polymers) and includes, for example, containers in the form of (i) a syringe, (ii) a vial, (iii) a carpule, or (iv) an ampoule. In a preferred embodiment of the present invention, the liquid formulation is stored in a syringe in the form of a prefilled syringe, as known in the art.

As to component (i), the botulinum toxin is not particularly limited and includes botulinum toxin of any serotype (BoNT/A-G). For example, the botulinum toxin may be of serotype A or B (BoNT/A, BONT/B). Preferably, the botulinum toxin is of serotype A, more preferably of serotype A1 (BoNT/A1), and most preferably BoNT/A1 produced by Clostridium botulinum Hall strain. In addition, the botulinum toxin may be a natural neurotoxin obtainable from the bacteria Clostridium botulinum or any other botulinum toxin such as a botulinum toxin obtainable from alternative sources, including recombinant technologies and genetic or chemical modification.

Furthermore, as used herein, the term “botulinum toxin” (“BT”) and the synonymously used term “botulinum neurotoxin” (“BONT”) are intended to refer to the pure botulinum neurotoxin and/or any complex thereof, i.e., any complex of the pure botulinum neurotoxin and complexing proteins (referred to as the “toxin complex”).

The term “pure botulinum neurotoxin”, as used herein, means the botulinum neurotoxin free of complexing proteins (sometimes also referred to as the “neurotoxic component”), or more precisely, the botulinum neurotoxin without neurotoxin-associated complexing proteins (NAPs). The pure botulinum neurotoxin is the (active) neurotoxic polypeptide that ultimately inhibits acetylcholine release. It is a di-chain protein comprised of a light chain (LC; about 50 kDa) and a heavy chain (HC; about 100 kDa), held together by a disulfide bond. The active neurotoxic polypeptide may therefore also be referred to herein as the “150 kDa neurotoxin”, “Clostridium botulinum neurotoxin (150 kD)” or “neurotoxic component”.

The term “toxin complex”, as used herein, refers to a high-molecular complex of the neurotoxic component and a set of complexing proteins (NAPs). In particular, the term “toxin complex” includes the 900 kDa, 500 kDa, and 300 kDa C. botulinum type A toxin complexes. The complexing proteins are nontoxic nonhaemagglutinin (NTNHA) and, in strains of serotypes A-D, different haemagglutinins (HAs). For example, the 900 kDa complex is included in onabotulinumtoxin A (Botox®/Vistabel®, Allergan, Inc., Irvine, CA, USA). Also, a toxin complex as active agent is contained in Dysport® (Azzalure®, Ipsen, Paris, France), Alluzience® (Ipsen/Galderma) and Innotox® (Medytox).

The concentration of the botulinum toxin in the liquid formulation of the present invention may be in the range of 1-1000 U/ml, and is preferably in the range of 10-200 U/ml, more preferably in the range of 20-150 U/ml, such as 50 U/ml or 100 U/ml.

The term “units” or “U”, as used herein refers to the biological activity (biological potency) of the toxin and relates to the dose that is lethal to 50% of mice tested (LD₅₀). More specifically, within the context of the present invention, the LD₅₀ is measured using the mouse bioassay (MBA), unless otherwise stated. The MBA determines the mean lethal dose (LD₅₀) of toxin/neurotoxin after intraperitoneal injection in mice, i.e., the dose of toxin/neurotoxin capable of killing 50% of a group of mice. On this basis, 1 unit (U) of toxin/neurotoxin, as used herein, is defined as one mouse LD₅₀ (1.0 LD_{50=1.0} U). The LD₅₀ mouse bioassay is the gold standard among various biological, chemical or immunological detection methods for botulinum toxin and is known to those skilled in the art (see, e.g., Pearce, L. B.; Borodic, G. E.; First, E. R.; MacCallum, R. D. Measurement of botulinum toxin activity: Evaluation of the lethality assay. Toxicol. Appl. Pharmacol. 1994, 128:69-77). A person skilled in the art will be able to determine suitable botulinum toxin concentrations depending on the serotype and the intended use.

Alternatively, also a cell-based assay can be used to determine botulinum toxin activity, as described in WO 2009/114748, WO 2013/049508 or WO2014/207109. A person skilled in the art will be able to correlate botulinum toxin activity results obtained with a cell-based assay with results obtained in the mouse LD₅₀ assay by calibration using a LD₅₀ reference standard.

Due to differences in the LD₅₀ tests used by manufacturers of commercial botulinum toxin formulations, the unit potencies indicated by the manufacturers for their commercial botulinum toxin formulations is proprietary and cannot easily be compared. Therefore, within the framework of the present invention, the conversion rates provided below are used to establish the comparative potencies of incobotulinumtoxinA (“INCO”; Xeomin®, Bocouture®; botulinum toxin serotype A, free of complexing proteins; Merz Pharmaceuticals GmbH), onabotulinumtoxinA (“ONA”; Botox®, Vistabel®; botulinum toxin complex of serotype A; Allergan Inc.), abobotulinumtoxinA (“ABO”; Dysport®, Azzalure®; botulinum toxin complex of serotype A; Medicis Pharmaceutical Corp., Galderma Lab.), rimabotulinumtoxinB (“RIM”; Myobloc® NeuroBloc®; botulinum toxin serotype B; Solstice Neurosciences Inc.), and PurTox® (“TBD”; botulinum toxin serotype A; Mentor Worldwide LLC). For use herein, the conversion rate of ONA and INCO is 1:1. The conversion rate of ONA/INCO: ABO is 1:2.5. The conversion rate of ONA/INCO: RIM is 1:50, and the conversion rate of ONA/INCO: TBD is 1:1.5.

As to component (ii) of the liquid formulation of the present invention, the term “stabilizing protein” generally refers to a polypeptide that results in an increased stability of the botulinum toxin. As used herein, the term “polypeptide” is interchangeably used with the term “protein” and refers to a peptide that consists of more than 10 amino acids, preferably more than 50 amino acids, more preferably more than 100 amino acids, and most preferably more than 200 amino acids, and up to preferably no more than 1000 amino acids, linked together by peptide bonds. Particularly preferred for use herein are polypeptides with 150 to 1000 amino acids or 250 to 800 amino acids, in particular 300 to 1000 amino acids or 350 to 700 amino acids.

Within the present invention, the stabilizing protein may be, for example, human serum albumin (HSA), ovalbumin, casein, or a mixture thereof. Particularly preferred for use herein is human serum albumin, ovalbumin, or a mixture thereof, and most preferred is HSA.

The stabilizing protein or mixture of stabilizing proteins may be present in the liquid formulation in an amount of 0.001-2.0% w/v, preferably in an amount of 0.01-1.0% w/v, more preferably in an amount of 0.01-0.5% w/v, and most preferably in an amount of 0.02-0.3% w/v or 0.03-0.10% w/v. The most preferred stabilizing protein, i.e., human serum albumin (HSA), is preferably present in the liquid formulation in an amount of 0.001-1.00% w/v, more preferably 0.01-0.3% w/v, and most preferably 0.02-0.15% w/v. As used herein, the term “human serum albumin” or “HSA” is intended to refer to donor HSA (HSA derived from human blood or, more precisely, from human plasma), recombinant HSA and/or HSA from any other source. Preferably, the human serum albumin is recombinant HSA or HSA derived from human blood.

Whilst not being bound by theory, it is thought that the light-sensitivity of the formulation of the present invention relates at least partly to HSA, or a component included in the used HSA material, since it was surprisingly found that the light stability decreases with increasing HSA concentrations.

As to component (iii), the chelating agent is not particularly limited as far as it is capable of binding metal ions. The term “chelating agent”, as used herein, may also be referred to as “chelator” or “sequestering agent”. The chelating agents for use herein are typically metal ion-binding organic compounds. The metal ions generally form multiple coordinate bonds with the organic chelating agents which act as polydentate ligands.

Suitable chelating agents for use herein include, but are not limited to, aminopolycarboxylic acids (e.g., aminopolycarboxylic acids having three to six, preferably four, carboxylic acid functional groups) and other compounds such as citrate, porphyrins, TPEN (N,N,N′,N′-tetrakis(2-pyridinylmethyl)-1,2-ethanediamine), TETA (triethylenetetramine), and mixtures thereof. Exemplary aminopolycarboxylic acids include NTA (nitrilotriacetate), DOTA (1,4,7,10-tetraazacyclododecan-1,4,7,10-tetraacetate), TED (ethylenediaminotriacetate), EDTA (ethylenediaminetetraacetic acid), EGTA (ethylene glycol-bis(β-aminoethylether)-N,N,N′,N′-tetraacetic acid), BAPTA (1,2-bis(o-aminophenoxy) ethane-N,N,N′,N′-tetraacetic acid), DTPA (diethylenetriaminepenta-acetic acid), and TTHA (triethylenetetraminehexaacetate).

Particularly preferred chelating agents for use herein include compounds of general formula (I):

(HO₂CCH₂)₂N—R—N(CH₂CO₂H)₂ (I),

which are aminopolycarboxylic acid compounds having at least four carboxylic acid functional groups. The R group is not particularly limited and may comprise no carboxylic acid functional group or one or two carboxylic acid functional groups. Preferably, the R group comprises no or one carboxylic acid functional group, most preferably no carboxylic acid functional group.

Examples of compounds of general formula (I) include, for example, EDTA, EGTA, BAPTA, DTPA, and TTHA. Particularly preferred for use herein are EDTA, EGTA, and DTPA, more preferred are EDTA and DTPA, and most preferred is EDTA. Mixtures of any of the above-mentioned chelating agents may also be used within the present invention.

The chelating agent may be present at a concentration of at least 0.01 mM or at least 0.1 mM or at least 1 mM. Preferably, the chelating agent is present at a concentration of 0.01-100 mM or at a concentration of 0.1-50 mM, more preferably at a concentration of 0.05-20 mM, and most preferably at a concentration of 1-10 mM.

Further to component (iii), it is contemplated herein that instead of, or in addition to, the chelating agent(s), phosphate may be used in the liquid formulation of the present invention to achieve a reduced light sensitivity of the formulation. In fact, it was unexpectedly found that also the presence of phosphate results in a markedly increased light stability. The phosphate may be present in the liquid formulation at a concentration of at least 0.1 mM or at least 1 mM or at least 5 mM. Preferably, the phosphate is present in the liquid formulation at a concentration of 0.1-100 mM, preferably 1-50 mM, more preferably 5-30 mM.

Preferably, the liquid formulation of the present invention comprises (i) botulinum toxin, (ii) human serum albumin, and (iii) a chelating agent, preferably EDTA. This preferred liquid formulation, like any other liquid formulation described herein above, may further comprise one or more of optional components (iv), (v) and (vi) described below, i.e., (iv), or (v), or (vi), or (iv) and (v), or (iv) and (vi), or (v) and (vi), or (iv) and (v) and (vi).

In a preferred embodiment of the present invention, the liquid formulation may further comprise a salt of an alkaline earth metal or a transition metal, and preferably further comprises:
(iv) a salt of calcium, magnesium or zinc, or a mixture thereof.

Preferred salts are salts of calcium, magnesium and zinc, more preferably salts of calcium and magnesium, most preferably a salt of calcium. In the presence of EDTA, the addition of these salts will result in the formation of EDTA complexes, i.e., EDTA with the added metal cation complexed as central atom. The nature of the anion of the salt is immaterial, the only requirement being that the salt is soluble in the aqueous formulation of the present invention. For example, the anion may be an anion of pharmaceutically acceptable inorganic and organic acids. Alternatively, the chelating agent itself can form the anion of the metal salt, and components (iii) and (iv) are added to the liquid formulation as, e.g., Na₂CaEDTA.

Suitable counter anions include, for example, acetate, aspartate, benzenesulfonate, benzoate, besylate, bicarbonate, bitartrate, bromide, camsylate, carbonate, chloride, citrate, decanoate, edetate, esylate, fumarate, gluceptate, gluconate, glutamate, glycolate, glycollylarsanilate, hexanoate, hydrabamine, hydroxynaphthoate, iodide, lactobionate, nitrate, malate, maleate, mandelate, mesylate, methylbromide, methylnitrate, methylsulfate, mucate, napsylate, nitrate, octanoate, oleate, palmoate, pantothenate, phosphate, polygalacturonate, propionate, salicylate, stearate, subacetate, succinate, sulfate, tartrate, and teoclate.

Preferably, the salts are chloride salts. Particularly preferred salts include calcium chloride (i.e., CaCl₂) and hydrates thereof), magnesium chloride (i.e., MgCl₂ and hydrates thereof) and zinc chloride (i.e., ZnCl₂ and hydrates thereof). In the presence of EDTA, the addition of these salts will result in the formation of EDTA complexes, i.e., EDTA with the added metal cation complexed as central atom.

Generally, it is envisaged that the concentration of the metal ion of the salt is about equimolar to the concentration of the chelating agent. The salt may be present in the liquid formulation at a concentration of at least 0.01 mM or at least 0.1 mM or at least 1 mM. Preferably, the salt is present in the liquid formulation at a concentration of 0.01-100 mM, preferably 0.1-50 mM, more preferably 0.05-20 mM, and most preferably 1-10 mM.

Surprisingly, it was found by the inventors of the present invention that the addition of a salt of an alkaline earth metal or a transition metal, in particular a salt of calcium, magnesium or zinc, markedly reduces the injection pain of the liquid formulation of the present invention while at the same time maintaining the storage and light stability. This finding was unexpected because (i) the liquid botulinum toxin formulations as described herein above were found to cause an unpleasant injection pain, which was neither foreseeable nor predictable, (ii) the addition of a salt of an alkaline earth metal or a transition metal such as calcium chloride was surprisingly found to drastically reduce the injection pain and, at the same time, (iii) the storage and light stability of the liquid formulation was maintained despite the belief that a free (non-complexed) chelating agent is required for obtaining the light stabilizing effect, i.e., a chelating agent (e.g., EDTA) containing a central metal atom (e.g., Ca²⁺) was surprisingly found to be still capable of protecting botulinum toxin from light.

In accordance with the present invention, the liquid formulation may optionally further comprise:
(v) a tonicity agent.

The term “tonicity agent”, as used herein, refers to an agent that is added to injectable formulations to render the formulations similar in osmotic characteristics to physiologic fluids. The tonicity agent may also be referred to as “osmotic regulator”. The tonicity agent is not particularly limited and may, for example, be selected from the group consisting of sugars, salts, polymers, and mixtures thereof.

Exemplary tonicity agents include sucrose, glucose, sodium carbonate, amino acids, polyethyleneglycol (PEG), dextran, cyclodextrin, and colloides (e.g., colloidal polysaccharides). Typically, the concentration of the tonicity agent is in the range of 0-2.0% w/v, in particular 0.01-2.0% w/v or 0.1-1.5% w/v, more particularly 0.6-1.2% w/v.

Preferably, the tonicity agent is sodium chloride (NaCl). The sodium chloride may be present in the liquid formulation of the present invention in an amount of 0.01-2.0% w/v, preferably 0.1-1.5% w/v, more preferably 0.5-1.2% w/v or 0.8-1.0% w/v, and most preferably 0.9% w/v.

In accordance with the present invention, the liquid formulation may optionally further comprise:
(vi) a buffering agent.

The term “buffering agent”, as used herein, means an agent which maintains the pH of the liquid formulation in an acceptable range, i.e., an agent capable of controlling the pH of the formulation. Suitable buffers are those that are not chemically reactive with other ingredients and are present in amounts sufficient to provide the desired degree of pH buffering. Such buffers include, for example, amino acids, acetate, malic acid, ascorbate, citrate, tartrate, fumarate, succinate, phosphate, bicarbonate, TRIS, Bis-TRIS, ACES, MES, BES, MOPS, HEPES, TES, PIPES, tricine, and imidazole.

Preferably the buffering agent is phosphate, an amino acid, or a mixture thereof. It is noted that the phosphate may play a dual role within the present invention, i.e., it may function as a phosphate pH buffer and also exert a stabilizing effect as component (iii) of the present invention. The amino acid may be selected from aspartate, glycine, glutamate, histidine, proline, taurine, methionine, serine, tyrosine, tryptophan, and mixtures thereof, and is preferably selected from histidine, proline, taurine, methionine, serine, tyrosine, tryptophan, and mixtures thereof. Most preferably, the amino acid is histidine. The most preferred buffering agent for use herein is histidine, phosphate or a mixture thereof.

The concentration of the buffering agent in the liquid formulation of the present invention is preferably 1-100 mM, more preferably 2-50 mM, and most preferably 5-20 mM. If the buffering agent is an amino acid (e.g., histidine), it may be present in the liquid formulation at a concentration of 1-100 mM, preferably 2-50 mM, more preferably 5-20 mM, and most preferably 10 mM. If the buffering agent is phosphate, it may be present in the liquid formulation at a concentration of 1-100 mM, preferably 2-50 mM, more preferably 5-20 mM, and most preferably 10 mM.

The pH of the liquid formulation of the present invention is typically in the range of 5.0-8.0, particularly in the range of 5.5-7.5, and preferably in the range of 5.5-7.0 or 6.0-7.5, more preferably in the range of 6.0-7.0, and most preferably in the range of 6.0-6.5.

Moreover, the liquid formulation of the present invention may further comprise one or more additional pharmaceutically acceptable excipients, unless otherwise stated or intended. For example, the liquid formulation may contain one or more of glycerol, sucrose, lactose, mannitol, hyaluronic acid, lactic acid, citric acid, amino acid(s), benzyl alcohol, lidocaine, gelatine, hydroxyethyl starch (HES), polyethyleneoxide, and polysorbate (e.g., polysorbate 20, polysorbate 80). Other suitable pharmaceutically acceptable excipients comprise those well known in the art, see, e.g., Remington's Pharmaceutical Sciences, Mack Publishing Company, Easton, Pennsylvania.

On the other hand, it is also contemplated herein that the liquid formulation of the present invention specifically lacks certain components (i.e., compounds, materials or substances), for example detergents, polysaccharides, amino acids, stabilizing peptides, and the like, including any combination thereof. As used herein, the term “detergent”, is synonymously used with “surfactant” and is intended to include non-ionic and ionic detergents. The term “stabilizing peptide”, as used herein, generally means a peptide consisting of 5 to 50 amino acids, such as a peptide of 10 to 40 amino acids or 15 to 30 amino acids. Hence, the term “stabilizing peptide” excludes HSA.

In one embodiment, the liquid formulation of the present invention does not contain detergents, particularly does not contain polysorbate, more particularly does not contain polysorbate 20 and/or polysorbate 80. In another embodiment, the liquid formulation of the present invention does not contain alginate. In another embodiment, the liquid formulation of the present invention does not contain succinate. In another embodiment, the liquid formulation of the present invention does not contain one or more (e.g., 2, 3, 4 or 5) amino acids selected from the group consisting of: arginine, glutamic acid, methionine, tryptophane, and serine. In another embodiment, the liquid formulation of the present invention does not contain a saccharide, such as a mono-, oligo- or polysaccharide or a mixture thereof. In particular, the liquid formulation of the present invention may not contain one or more (e.g., 2, 3 or 4) of sucrose, lactose, maltose, and trehalose. It is also contemplated within the present invention that the liquid formulation lacks more than one, or all of, the compounds mentioned above.

In one embodiment, the liquid formulation of the present invention lacks (i) detergents and mono-, oligo- and polysaccharides, (ii) detergents and any amino acid, or detergents and all amino acids except histidine, (iii) detergents and stabilizing peptides, (iv) mono-, oligo- and polysaccharides and any amino acid, or mono-, oligo- and polysaccharides and all amino acid except histidine, (v) mono-, oligo- and polysaccharides and stabilizing peptides, (vi) any amino acids and stabilizing peptides, or all amino acids except histidine and stabilizing peptides, (vii) detergents, mono-, oligo- and polysaccharides and any amino acid, or detergents, mono-, oligo- and polysaccharides and all amino acids except histidine, (viii) detergents, mono-, oligo- and polysaccharides, and stabilizing peptides, (ix) detergents, any amino acid, and stabilizing peptides, or detergents, all amino acid except histidine, and stabilizing peptides, (x) mono-, oligo- and polysaccharides, any amino acid, and stabilizing peptides, or mono-, oligo- and polysaccharides, all amino acids except histidine, and stabilizing peptides, (xi) detergents, mono-, oligo- and polysaccharides, any amino acid, and stabilizing peptides, or detergents, mono-, oligo- and polysaccharides, all amino acids except histidine, and stabilizing peptides.

In another embodiment, the liquid formulation of the present invention does not contain any other amino acid than histidine. In another embodiment, the liquid formulation of the present invention does not contain any mono-, di- and trisaccharides. In another embodiment, the liquid formulation of the present invention does not contain any other stabilizing peptide or protein than HSA. In another embodiment, the liquid formulation of the present invention does not contain phosphate such as in the form of a phosphate buffer.

In accordance with preferred embodiments of the present invention, the liquid formulation lacks (i) succinate and a detergent (e.g., polysorbate), (ii) succinate and methionine, (iii) succinate and sucrose, (iv) a detergent (e.g., polysorbate) and methionine, (v) a detergent (e.g., polysorbate) and sucrose, (vi) methionine and sucrose, (vii) succinate, a detergent (e.g., polysorbate) and methionine, (xiii) succinate, a detergent (e.g., polysorbate) and sucrose, (ix) succinate, methionine and sucrose, (x) a detergent (e.g., polysorbate), methionine and sucrose, and (xi) succinate, a detergent (e.g., polysorbate), methionine and sucrose, (xii) a detergent (e.g. polysorbate and histidine), (xiii) a detergent (e.g. polysorbate), histidine and sucrose).

A preferred liquid formulation of the present invention comprises (i) botulinum toxin, (ii) HSA, and (iii) a chelating agent of general formula (HO₂CCH₂)₂N—R—N(CH₂CO₂H)₂, wherein R comprises no carboxylic acid group or one or two carboxylic acid groups. A preferred liquid formulation of the present invention comprises (i) botulinum toxin at a concentration of 10-200 U/ml, (ii) HSA in an amount of 0.01-0.5% w/v, and (iii) a chelating agent of general formula (HO₂CCH₂)₂N—R—N(CH₂CO₂H)₂, wherein R comprises no carboxylic acid group or one or two carboxylic acid groups, at a concentration of 0.05-20 mM.

A preferred liquid formulation of the present invention comprises (i) botulinum toxin, (ii) HSA, and (iii) a chelating agent selected from the group consisting of DOTA, TED, EDTA, EGTA, BAPTA, DTPA and TTHA or a chelating agent selected from the group consisting of EDTA, EGTA, BAPTA, DTPA and TTHA. A preferred liquid formulation of the present invention comprises (i) botulinum toxin at a concentration of 10-200 U/ml, (ii) HSA in an amount of 0.01-0.5% w/v, and (iii) a chelating agent selected from the group consisting of DOTA, TED, EDTA, EGTA, BAPTA, DTPA and TTHA or a chelating agent selected from the group consisting of EDTA, EGTA, BAPTA, DTPA and TTHA at a concentration of 0.05-20 mM.

A preferred liquid formulation of the present invention comprises (i) botulinum toxin, (ii) HSA, and (iii) EDTA. A preferred liquid formulation of the present invention comprises (i) botulinum toxin at a concentration of 10-200 U/ml, (ii) HSA in an amount of 0.01-0.5% w/v, and (iii) EDTA at a concentration of 0.05-20 mM.

A preferred liquid formulation of the present invention comprises (i) botulinum toxin, (ii) HSA, (iii) a chelating agent of general formula (HO₂CCH₂)₂N—R—N(CH₂CO₂H)₂, wherein R comprises no carboxylic acid group or one or two carboxylic acid groups, and (iv) a salt of calcium, magnesium or zinc, or a mixture thereof, preferably calcium chloride. A preferred liquid formulation of the present invention comprises (i) botulinum toxin at a concentration of 10-200 U/ml, (ii) HSA in an amount of 0.01-0.5% w/v, (iii) a chelating agent of general formula (HO₂CCH₂)₂N—R—N(CH₂CO₂H)₂, wherein R comprises no carboxylic acid group or one or two carboxylic acid groups, at a concentration of 0.05-20 mM, and (iv) a salt of calcium, magnesium or zinc, or a mixture thereof, preferably calcium chloride, at a concentration of 0.05-20 mM.

A preferred liquid formulation of the present invention comprises (i) botulinum toxin, (ii) HSA, (iii) a chelating agent selected from the group consisting of DOTA, TED, EDTA, EGTA, BAPTA, DTPA and TTHA or a chelating agent selected from the group consisting of EDTA, EGTA, BAPTA, DTPA and TTHA, and (iv) a salt of calcium, magnesium or zinc, or a mixture thereof, preferably calcium chloride. A preferred liquid formulation of the present invention comprises (i) botulinum toxin at a concentration of 10-200 U/ml, (ii) HSA in an amount of 0.01-0.5% w/v, (iii) a chelating agent selected from the group consisting of DOTA, TED, EDTA, EGTA, BAPTA, DTPA and TTHA or a chelating agent selected from the group consisting of EDTA, EGTA, BAPTA, DTPA and TTHA at a concentration of 0.05-20 mM, and (iv) a salt of calcium, magnesium or zinc, or a mixture thereof, preferably calcium chloride, at a concentration of 0.05-20 mM.

A preferred liquid formulation of the present invention comprises (i) botulinum toxin, (ii) HSA, (iii) EDTA, and (iv) a salt of calcium, magnesium or zinc, or a mixture thereof, preferably calcium chloride. A preferred liquid formulation of the present invention comprises (i) botulinum toxin at a concentration of 10-200 U/ml, (ii) HSA in an amount of 0.01-0.5% w/v, (iii) EDTA at a concentration of 0.05-20 mM, and (iv) a salt of calcium, magnesium or zinc, or a mixture thereof, preferably calcium chloride, at a concentration of 0.05-20 mM.

A preferred liquid formulation of the present invention comprises (i) botulinum toxin, (ii) HSA, (iii) a chelating agent of general formula (HO₂CCH₂)₂N—R—N(CH₂CO₂H)₂, wherein R comprises no carboxylic acid group or one or two carboxylic acid groups, (iv) a salt of calcium, magnesium or zinc, or a mixture thereof, preferably calcium chloride, and (v) a phosphate buffer and/or histidine. A preferred liquid formulation of the present invention comprises (i) botulinum toxin at a concentration of 10-200 U/ml, (ii) HSA in an amount of 0.01-0.5% w/v, (iii) a chelating agent of general formula (HO₂CCH₂)₂N—R—N(CH₂CO₂H)₂, wherein R comprises no carboxylic acid group or one or two carboxylic acid groups, at a concentration of 0.05-20 mM, (iv) a salt of calcium, magnesium or zinc, or a mixture thereof, preferably calcium chloride, at a concentration of 0.05-20 mM, and (v) a phosphate buffer and/or histidine at a concentration of 2-50 mM.

A preferred liquid formulation of the present invention comprises (i) botulinum toxin, (ii) HSA, (iii) a chelating agent selected from the group consisting of DOTA, TED, EDTA, EGTA, BAPTA, DTPA and TTHA or a chelating agent selected from the group consisting of EDTA, EGTA, BAPTA, DTPA and TTHA, (iv) a salt of calcium, magnesium or zinc, or a mixture thereof, preferably calcium chloride, and (v) a phosphate buffer and/or histidine. A preferred liquid formulation of the present invention comprises (i) botulinum toxin at a concentration of 10-200 U/ml, (ii) HSA in an amount of 0.01-0.5% w/v, (iii) a chelating agent selected from the group consisting of DOTA, TED, EDTA, EGTA, BAPTA, DTPA and TTHA or a chelating agent selected from the group consisting of EDTA, EGTA, BAPTA, DTPA and TTHA at a concentration of 0.05-20 mM, (iv) a salt of calcium, magnesium or zinc, or a mixture thereof, preferably calcium chloride, at a concentration of 0.05-20 mM, and (v) a phosphate buffer and/or histidine at a concentration of 2-50 mM.

A preferred liquid formulation of the present invention comprises (i) botulinum toxin, (ii) HSA, (iii) EDTA, (iv) a salt of calcium, magnesium or zinc, or a mixture thereof, preferably calcium chloride, and (v) a phosphate buffer and/or histidine. A preferred liquid formulation of the present invention comprises (i) botulinum toxin at a concentration of 10-200 U/ml, (ii) HSA in an amount of 0.01-0.5% w/v, (iii) EDTA at a concentration of 0.05-20 mM, (iv) a salt of calcium, magnesium or zinc, or a mixture thereof, preferably calcium chloride, at a concentration of 0.05-20 mM, and (v) a phosphate buffer and/or histidine at a concentration of 2-50 mM.

Particularly preferred, the above-described preferred liquid formulations further contain sodium chloride, in particular sodium chloride at a concentration of about 0.9% w/v. Furthermore, the pH is preferably in the range of 6.0-7-5. Moreover, the botulinum toxin is preferably of serotype A and is, particularly, the neurotoxic component of serotype A.

Exemplary preferred liquid formulations of the present invention include the following:

### Formulation 1:

50 U/ml BoNT/A
0.9% NaCl (9 mg/mL)
0.05% HSA (0.5 mg/mL)
0.1% Na₂-EDTA (1 mg/ml; ˜2.7 mM),
pH 5.5-8.0 (e.g., 6.0), and optionally
a pH buffer (e.g., 10 mM phosphate or 10 mM histidine)

### Formulation 2:

50 U/ml BoNT/A
0.9% NaCl (9 mg/mL)
0.085% HSA (0.85 mg/mL)
0.155% histidine (1.55 mg/ml; ˜10 mM)
0.13% Na₂-EDTA (1.3 mg/ml; ˜3.5 mM)
0.039% CaCl₂) (390 μg/mL; ˜3.5 mM)
pH 5.5-8.0 (e.g., 6.0), and optionally
a pH buffer (e.g., 10 mM phosphate)

The present invention also relates to a liquid formulation comprising a botulinum toxin, wherein the toxin activity is not reduced by more than 35%, relative to the initial toxin activity, upon storage of the liquid formulation for 4 weeks at an elevated temperature of 40° C. In another aspect, the present invention relates to a liquid formulation comprising a botulinum toxin, wherein the toxin activity is not reduced by more than 35%, relative to the initial toxin activity, upon exposure of the liquid formulation for 7 hours to a light source at 250 W/m².

Moreover, the present invention further relates to a method for preparing the liquid botulinum toxin formulation described herein, comprising combining the component described herein, in particular components (i) to (iii), and optionally one or more of components (iv) to (vi). The preparation of the liquid formulation of the present invention is not particularly limited and the respective techniques are known to those skilled in the art. As described above, the liquid formulation of botulinum toxin is generally an aqueous solution, preferably a saline solution, more preferably a physiological saline solution, and most preferably a buffered (e.g., phosphate or histidine buffered) physiological saline solution.

Preferably, the salts are dissolved first, then HSA is added, the pH is adjusted if necessary, and finally the botulinum toxin is added. This order is not a mandatory requirement but is believed to safely preserve maximum specific activity of the botulinum toxin. Preferably, the method for preparing the liquid botulinum toxin formulation does not comprise the reconstitution of a lyophilized botulinum toxin preparation in powder form.

Further, the present invention relates to a method for stabilizing a liquid botulinum toxin formulation, comprising combining (i) botulinum toxin, (ii) a stabilizing protein, preferably human serum albumin (HSA), (iii) a chelating agent or phosphate, and optionally one or more of components (iv) to (vi), wherein components (i) to (v) are as defined herein.

Still further, the present invention relates to a use of a chelating agent as defined herein for increasing the light stability of an aqueous botulinum toxin formulation containing (i) botulinum toxin and (ii) a stabilizing protein, preferably human serum albumin, and optionally one or more of components (iv) to (vi), wherein components (i), (ii), and (iv) to (vi) are as defined herein. An “increase in light stability” in this context refers to an aqueous botulinum toxin formulation containing a chelating agent, which has a biological botulinum toxin activity after exposure to light at 250 W/m² for 7 hours at a temperature (e.g., 20° C.) that is at least 20% higher, preferably at least 40% higher, more preferably at least 60% higher, and most preferably at least 80% higher than that of the aqueous botulinum toxin formulation without the chelating agent after exposure to light at 250 W/m² for 7 hours at the same temperature (e.g., 20° C.).

Yet further, the present invention relates to a use of phosphate for increasing the light stability of an aqueous botulinum toxin formulation containing (i) botulinum toxin and (ii) a stabilizing protein, preferably human serum albumin, and optionally one or more of components (iv) to (vi), wherein components (i), (ii), and (iv) to (vi) are as defined herein. An “increase in light stability” in this context refers to an aqueous botulinum toxin formulation containing phosphate, which has a biological botulinum toxin activity after exposure to light at 250 W/m² for 7 hours at a temperature (e.g., 20° C.) that is at least 10% higher, preferably at least 20% higher, more preferably at less 30% higher, and most preferably at least 40% higher than that of the aqueous botulinum toxin formulation without phosphate after exposure to light at 250 W/m² for 7 hours at the same temperature (e.g., 20° C.).

Furthermore, the present invention relates to the use of a salt of calcium, magnesium or zinc, or a mixture thereof, for reducing injection pain of a botulinum toxin formulation that comprises (i) botulinum toxin, (ii) a stabilizing protein, preferably human serum albumin (HSA), (iii) a chelating agent or phosphate, and optionally one or more of components (iv) to (vi), wherein components (i) to (vi) are as defined herein.

In another aspect, the present invention relates to a liquid formulation of the present invention for use in therapy.

In particular, the liquid formulation of the present invention may be used in the treatment of neuromuscular diseases, pain, sialorrhea, hyperhidrosis, urological disorders, and neurological disorders. Exemplary neuromuscular diseases include dystonia, cervical dystonia, spasm, post-stroke spasticity, blepharospasm, tremor, hyperkinetic movement disorders, and cerebral palsy. The urological disorders include, among others, conditions characterized by detrusor overactivity, overactive bladder, neurogenic bladder and interstitial cystitis, treatment of vulvodynia and chronic pelvic pain, benign prostate hyperplasia (BPH) and detrusor sphincter dyssynergia (DSD). Exemplary neurological disorders include chronic migraines, trigeminal pain, peripheral neuropathic pain, diabetic neuropathic pain and depression.

In yet another aspect, the present invention relates to a cosmetic use of the liquid formulation of the present invention for the treatment of a cosmetic condition.

This aspect of the present invention relates to a purely aesthetic use of the liquid formulation of the present invention. Preferred cosmetic conditions to be treated include skin conditions, in particular the treatment of wrinkles of the skin, in particular of the face.

The term “wrinkles”, as used herein, is to be broadly construed to not only include wrinkles, but also lines, rhytids, creases, furrows, and folds. The words “lines”, “wrinkles”, “rhytids”, “creases”, and “folds” share similar definition and are therefore often used interchangeably. Within the present invention, “lines” are generally interchangeable with “wrinkles” but may preferably refer to a cutaneous depression that is less deep than a “wrinkle”. A “fold” is interchangeable with wrinkles and lines and is preferably a linear depression. A “crease” is interchangeable with wrinkles, lines and folds. It preferably refers to a mild form of wrinkles and may describe the specific wrinkle in certain locations. A “rhytid”, as used herein, has essentially the same meaning of wrinkle. However, a “rhytid” preferably refers to a skin structure that is formed by irregular aggregation of lines. A “furrow” is a deep fold or deep line in the skin.

Preferably, the wrinkles treated in accordance with the present invention are facial wrinkles including include horizontal forehead lines, glabellar frown lines, periorbital lines, Crow's feet, bunny lines (i.e., downward radiating lines on the sides of nose), nasolabial folds, upper radial lip lines, lower radial lip lines, corner of the mouth lines, marionette lines, perioral lip lines, oral commissures, labiomental crease and cobblestone chin.

In order to treat the above-mentioned facial wrinkles, botulinum toxin is usually administered by intramuscular injection to the following muscles: frontalis muscle (horizontal forehead lines), procerus and corrugator muscles (glabellar frown lines), lateral orbicularis oculi muscle (Crow's feet/periorbital lines), nasalis, procerus for transverse nasal muscles (bunny lines), levator labii superioris alaeque nasi (nasolabial folds), orbicularis oris (upper and lower radial lip lines), depressor anguli oris (corner of the mouth lines, marionette lines, oral commissures, labiomental crease), and mentalis muscles (perioral lip lines, cobblestone chin).

A further preferred cosmetic use of the liquid formulation of the present invention relates to the use for cosmetic applications comprising rejuvenation and/or improvement of the skin quality of the face and/or body.

In still a further aspect, the present invention relates to a method of treating a disease or condition, comprising administering an effective amount of the liquid formulation of the present invention to a person in need thereof.

The disease or condition may be any one of the diseases and conditions mentioned hereinabove, irrespective of whether it is a therapeutic or cosmetic indication. Thus, in one embodiment, the present invention relates to a (non-therapeutic) method of treating a cosmetic (aesthetic) condition, preferably a skin condition, comprising administering an effective amount of the liquid formulation of the present invention to a person in need thereof. In another embodiment, the present invention relates to a (non-therapeutic) method of treating a cosmetic (aesthetic) condition, preferably a skin condition, comprising injecting an effective amount of the liquid formulation of the present invention into a person in need thereof.

A further preferred method of the present invention relates to a method of rejuvenation and/or improvement of the skin quality of the face and/or body, comprising administering an effective amount of the liquid formulation of the present invention to a person in need thereof.

The person to be treated is not particularly limited other than by having a disease or condition that can be treated in accordance with the present invention. Those skilled in the art will be able to determine appropriate administration regimens for the treatment of a given therapeutic or cosmetic indication. In particular, the injection may be intradermal, subdermal (subcutaneous), or intramuscular, depending on the disease or condition to be treated.

### EXAMPLES

The following Examples illustrate the liquid botulinum toxin formulation according to the present invention. Percentages are weight by volume (w/v) unless otherwise indicated.

### Example 1

### Heat Stability

The heat stability of botulinum toxin was determined by measuring the change in biological activity of botulinum toxin over time at an elevated temperature of 40° C. The biological activity was determined using a cell-based potency assay (CBA) as described in WO 2013/049508 and WO 2014/207109.

In brief, neuronal cells were incubated with the neurotoxin containing sample and a reference standard of known potency. After the incubation period, the cells were lyzed and the amount of cleaved SNAP25 protein was determined by an immunoassay. The biological activity of the sample is then calculated by comparing the cleavage rate of the cells treated with the sample with those treated with the reference standard.

A HSA-containing composition (“HSA composition”; 50 U/ml BoNT/A, 0.1% HSA, 0.47% saccharose, 0.9% NaCl) was compared with comparative composition 1 (50 U/ml BoNT/A, 0.015% polysorbate 20, 0.02% methionine, 0.9% NaCl, 0.078% NaH₂PO₄; pH 6.5) and comparative composition 2 (50 U/ml BoNT/A, 0.01% polysorbate 80, 0.155% histidine, 0.4% saccharose, 0.9% NaCl; pH 6.5). One portion of each composition was stored in the dark at 2-8° C. and analyzed in the CBA within 7 days (“T0”). The remaining portions of the compositions were stored at elevated temperature (40° C.) for 2 weeks prior to determining the biological activity. The botulinum toxin used in all compositions was the 150 kDa BoNT/A neurotoxin without complexing proteins. The results are shown in Table 1.

TABLE 1
Heat stability

| | Change in BoNT/A activity after |
|---|---|
| BoNT/A composition | 2 weeks at 40° C. relative to T0 |
| HSA composition (non-inventive) | −20% |
| Comparative composition 1 | −49% |
| Comparative composition 2 | −46% |

As can be seen from Table 1, the HSA composition containing human serum albumin (HSA) is significantly more stable than comparative compositions 1 and 2 that both lack HSA as stabilizing protein. Thus, the presence of a stabilizing protein such as HSA is important for achieving a sufficiently high toxin heat stability.

The impact of HSA on the stability of botulinum toxin was further studied by measuring the biological activity of BoNT/A (150 kDa) in liquid compositions with different HSA concentrations (75 U/ml BoNT/A, 0.03-1.0 mg/ml HSA, 10 mM phosphate, 10 mM histidine, 0.9% NaCl; pH 6.0) directly after the production process (at T0 as described above; T0 was the same for all studied samples). The results are shown in Table 2.

TABLE 2
Effect of HSA concentration on BoNT/A activity

| BoNT/A | | |
|---|---|---|
| composition | HSA | BoNT/A |
| (non-inventive) | concentration | activity |
| 1 | 0.03 mg/ml | 48 U/ml |
| 2 | 0.1 mg/ml | 63 U/ml |
| 3 | 0.3 mg/ml | 65 U/ml |
| 4 | 1.0 mg/ml | 75 U/ml |

As can be seen from Table 2, the toxin activity increases with increasing HSA concentrations.

### Example 2

### Light Sensitivity

When investigating the stability of various liquid formulations such as those described above, it was surprisingly discovered that light has a significant effect on the toxin stability. This discovery was further investigated by photostability testing using a SUNTEST CPS+ instrument (ATLAS Material Testing Technology LLC), equipped with a filter set to provide a spectral distribution in the wavelength range of 320-800 nm corresponding to the ID65 (indoor indirect daylight standard) per ISO 10977 with a window glass filter according to ICH Q1B. Photostability testing was carried out for 7 hours at 250 at 250 W/m².

Specifically, BoNT/A (150 kDa) compositions comprising buffered 0.9% NaCl (pH 6.0) and different concentrations of human serum albumin (0.03-1.0 mg/ml HSA) were prepared and then exposed to light for 7 hours at 250 W/m². For comparison, identical BoNT/A (150 kDa) compositions with different concentrations of HSA (0.03 mg/mL, 0.1 mg/mL, 0.3 mg/mL, and 1.0 mg/mL) were stored in the dark for the same period of time (control). Thereafter, the biological activity was measured for the compositions exposed to light and the respective control compositions stored in the dark. The relative BoNT/A activity expressed as percentage of the respective control was then calculated as a measure of light stability. The results are shown in Table 3.

TABLE 3
Effect of HSA on light stability

| BoNT/A | HSA | Relative BoNT/A |
|---|---|---|
| composition | concentration | activity (% of control) |
| 1 | 0.03 mg/ml | 82% |
| 2 | 0.1 mg/ml | 72% |
| 3 | 0.3 mg/ml | 70% |
| 4 | 1.0 mg/ml | 55% |

The results of Table 3 show that the light stability of BoNT/A in HSA-containing compositions decreases with increasing HSA concentration. Thus, while the heat stability increases with increasing HSA concentrations (see Example 1), it was unexpectedly found that HSA has a negative impact on light stability. Further studies (results not shown) revealed that the use of an alternative HSA product (recombinant HSA instead of donor HSA) or extensive dialysis of the HSA to remove potential small molecule impurities (<10 kDa) do not change these findings, i.e., have no impact on light sensitivity.

### Example 3

### Light Stability

Further investigations were carried out to find HSA-containing compositions with lower light sensitivity, i.e., higher light stability. These investigations included the preparation of a liquid composition comprising BoNT/A (150 kDa) without complexing proteins, 0.1% HSA, 0.47% saccharose, and 0.9% NaCl (composition “1”). In addition, two liquid compositions were prepared that correspond to composition 1, except that they further contain 2.7 mM EDTA (composition “1+EDTA”) and 20 mM phosphate (composition “1+Phosphate”; pH 6.0), respectively. Furthermore, a composition was prepared containing DTPA (composition “2 (DTPA)”), comprising BoNT/A (150 kDa), 0.01% HSA, 0.015% polysorbate 20, 10 mM phosphate, 0.155% histidine, 2.7 mM DTPA, 0.9% NaCl; pH 6.0).

Compositions 1, 1+EDTA, 1+Phosphate, and 2 (DTPA) were exposed to light for 7 hours at 250 W/m² as described above. In parallel, identical compositions were also stored at the dark for the same period of time (control). Then, the biological BoNT/A activity was measured in comparison to the respective control composition stored in the dark. The results are shown in Table 4.

TABLE 4
Effect of EDTA and DTPA on light stability

| BoNT/A | Relative BoNT/A |
|---|---|
| composition | activity (% of control) |
| 1 | 35% |
| 1 + EDTA | 77% |
| 1 + Phosphate | 60% |
| 2 (DTPA) | 77% |

As is evident from Table 4, the addition of the chelating agents EDTA and DTPA lead to a significant improvement in the light stability of HSA-containing BoNT/A compositions. It was also found that phosphate exerts a similar but weaker positive effect.

### Example 4

### Light Stability and Storage Stability in the Presence of Chelate Complexes

In following experiments, it was unexpectedly found that a liquid formulation of the present invention (0.9% NaCl, 3.5 mM EDTA, 10 mM histidine, 0.085% HSA, pH 6.0) caused a relatively strong injection pain when injected subcutaneously. Even very small amounts of EDTA (100 μl at a concentration of only 2.7 mM s.c.) already caused a very unpleasant pain. Various controls were used to exclude the possibility that the pain was a placebo effect or caused by the pH. Histidine could also be ruled out as a cause. It turned out that the burning pain was due to the complexing agent EDTA (results not shown). This finding was not expected in view of the fact that disodium EDTA is used in the treatment of heavy metal poisoning, in chelation therapy and in the formulation of vaccines.

Surprisingly, however, it was found that the addition of calcium or magnesium ions to the liquid botulinum toxin formulation markedly reduced the injection pain while the protective effect towards light was maintained. Also, a positive effect on storage stability at 40° C. was not only obtained by the addition of EDTA but also by the addition of EDTA complexes (EDTA and magnesium, calcium or zinc ions).

More specifically, in the experiments carried out, the following five different liquid compositions of BoNT/A 150 kDa (without complexing proteins) were compared:
Composition 1:0.103% HSA, 20 mM histidine, 0.9% NaCl, pH 6.0
Composition 2:0.085% HSA, 10 mM histidine, 0.9% NaCl, 3.5 mM EDTA, pH 6.0
Composition 3:0.085% HSA, 10 mM histidine, 0.9% NaCl, 3.5 mM EDTA, 3.5 mM MgCl₂, pH 6.0
Composition 4:0.085% HSA, 10 mM histidine, 0.9% NaCl, 3.5 mM EDTA, 3.5 mM ZnCl₂, pH 6.0.
Composition 5:0.085% HSA, 10 mM histidine, 0.9% NaCl, 3.5 mM EDTA, 3.5 mM CaCl₂), pH 6.0.

The relative BoNT/A activity was determined after exposure to light (7 hours at 250 W/m²; see above) relative to a control sample stored in the dark. The results are shown in Table 5.

TABLE 5
Light stability (7 h, 250 W/m²) of different
liquid botulinum toxin compositions

| BoNT/A | | Relative BoNT/A activity |
|---|---|---|
| composition | EDTA | (% of control (T0)) |
| 1 | w/o EDTA | 46% |
| 2 | Na—EDTA | 80% |
| 3 | Na—Mg—EDTA | 73% |
| 4 | Na—Zn—EDTA | 70% |
| 5 | Na—Ca—EDTA | 75% |

As can be seen, the compositions containing EDTA are significantly more stable against light than a composition without EDTA. The magnesium (Mg²⁺), calcium (Ca²⁺) and zinc (Zn²⁺) complexed metal ions only have a minor influence on the stabilizing properties.

These results are surprising because the complexing properties of free EDTA were believed to be essential for protecting BoNT/A against the destabilizing effects of light exposure, but the concentration of free EDTA were very low in the above experiments (e.g., for zinc in the range of 100 pM). However, contrary to expectation, the addition of metal ions such as magnesium and zinc did not abolish the light protective effect of EDTA.

Moreover, the relative BoNT/A activity was determined after storage at 40° C. for 2 and 4 weeks relative to a control sample at T0 (immediately after preparation of the compositions). The results are shown in Table 6.

TABLE 6
Storage stability at 40° C. of different
liquid botulinum toxin compositions

| | | Relative BoNT/A activity | |
|---|---|---|---|
| | | (% of control (T0)) | |
| BoNT/A | | after storage for 2 | after storage for 4 |
| composition | EDTA | weeks at 40° C. | weeks at 40° C. |
| 1 | w/o EDTA | 79% | 61% |
| 2 | Na—EDTA | 89% | 77% |
| 3 | Na—Mg—EDTA | 84% | 75% |
| 4 | Na—Zn—EDTA | 85% | 73% |
| 5 | Na—Ca—EDTA | 79% | 69% |

The results show that formulations with EDTA are more stable when stored at 40° C. than formulations without EDTA. Unexpectedly, the results further show that magnesium and zinc ions as central metal ions of an EDTA complex have little effect on the stabilizing properties.

1. A liquid formulation, comprising
(i) botulinum toxin,
(ii) a stabilizing protein, and
(iii) a chelating agent or phosphate.

2. The liquid formulation of claim 1, wherein the stabilizing protein is selected from human serum albumin, ovalbumin, casein, and mixtures thereof, or wherein the stabilizing protein is human serum albumin.

3. The liquid formulation of claim 1 or 2, wherein the stabilizing protein is human serum albumin and the human serum albumin is present in the liquid formulation at a concentration of 0.001-1.00% w/V.

4. The liquid formulation of any one of claims 1 to 3, wherein the chelating agent is selected from the group consisting of aminopolycarboxylic acids having three to six carboxylic acid functional groups, citrate, porphyrins, N,N,N′,N′-tetrakis(2-pyridinylmethyl)-1,2-ethanediamine (TPEN), triethylenetetramine (TETA), and mixture thereof.

5. The liquid formulation of claim 4, wherein the chelating agent is an aminopolycarboxylic acid of general formula (I):
(HO₂CCH₂)₂N—R—N(CH₂CO₂H)₂ (I),
wherein R comprises no carboxylic acid group or one or two carboxylic acid groups, the aminopolycarboxylic acid preferably being selected from the group consisting of ethylenediaminetetraacetic acid (EDTA), ethylene glycol-bis(β-aminoethylether)-N,N,N′,N′-tetraacetic acid (EGTA), 1,2-bis(o-aminophenoxy) ethane-N,N,N′,N′-tetraacetic acid (BAPTA), diethylenetriaminepentaacetic acid (DTPA), triethylenetetraminehexaacetate (TTHA), and mixture thereof.

6. The liquid formulation of any one of claims 1 to 5, wherein the chelating agent is present in the liquid formulation at a concentration of 0.01-100 mM.

7. The liquid formulation of any one of claims 1 to 6, wherein the botulinum toxin is botulinum neurotoxin free of complexing proteins, or wherein the botulinum toxin is of serotype A, or wherein the botulinum toxin is botulinum neurotoxin of serotype A free of complexing proteins, or wherein the botulinum toxin is of serotype A and is present at a concentration of 1-1000 U/ml, or wherein the botulinum toxin is botulinum neurotoxin of serotype A free of complexing proteins and is present at a concentration of 1-1000 U/ml.

8. The liquid formulation of any one of claims 1 to 7, comprising
(i) botulinum toxin,
(ii) human serum albumin, and
(iii) EDTA.

9. The liquid formulation of any one of claims 1 to 8, wherein the liquid formulation further comprises:
(iv) a salt of calcium, magnesium or zinc, or a mixture thereof,
or wherein the liquid formulation further comprises:
(iv) a salt of calcium, magnesium or zinc, or a mixture thereof, wherein the salt is present in the liquid formulation in an amount of 0.01-100 mM, wherein the salt is calcium chloride and is present in the liquid formulation in an amount of 0.01-100 mM.

10. The liquid formulation of any one of claims 1 to 9, wherein the liquid formulation further comprises:
(v) a tonicity agent,
or wherein the liquid formulation further comprises:
(v) a tonicity agent, wherein the tonicity agent is present in the liquid formulation in an amount of 0.01-2.0% w/v, or wherein the tonicity agent is sodium chloride, or wherein the tonicity agent is sodium chloride and is present in the liquid formulation in an amount of 0.01-2.0% w/v.

11. The liquid formulation of any one of claims 1 to 10, wherein the liquid formulation further comprises:
(vi) a buffering agent,
or wherein the liquid formulation further comprises:
(vi) a buffering agent, wherein the buffering agent is present in the liquid formulation in an amount of 1-100 mM, or wherein the buffering agent is an amino acid, phosphate, or a mixture thereof, or wherein the buffering agent is histidine, or wherein the buffering agent is histidine, phosphate, or a mixture thereof, and the histidine and phosphate are present in the liquid formulation in an amount of 1-100 mM.

12. The liquid formulation of any one of claims 1 to 11, wherein the pH of the liquid formulation is in the range of 5.0-8.0.

13. A liquid formulation according to any one of claims 1 to 12 for use in therapy, particularly for use in the treatment of neuromuscular diseases, pain, sialorrhea, hyperhidrosis, urological disorders, and neurological disorders.

14. Cosmetic use of the liquid formulation of any one of claims 1 to 12 for the treatment of a cosmetic condition.

15. A method of treating a disease or condition, comprising administering an effective amount of the liquid formulation of any one of claims 1 to 12 to a person in need thereof.
